(19) United States
(12) Patent Application Publication
(10) Pub. No.: US 2015/0173396 A1
(43) Pub. Date: Jun. 25, 2015

(54) CASEIN PRODUCTS AND C02 REVERSIBLE ACIDIFICATION METHODS USED FOR THEIR PRODUCTION

(71) Applicant: MURRAY GOULBURN CO-OPERATIVE CO. LIMITED, Victoria, (AU)

(72) Inventors: Darren Gardiner, Werribee (AU); Peter Hobman, Melbourne (AU)

(73) Assignee: MURRAY GOULBURN CO-OPERATIVE CO. LIMITED, Victoria, (AU)

(21) Appl. No.: 14/408,945

(22) PCT Filed: Jun. 20, 2013
(86) PCT No.: PCT/AU2013/000660

§ 371 Date: Dec. 17, 2014

(30) Foreign Application Priority Data
Jun. 20, 2012 (AU) 2012902595

(51) Int. Cl. A23J 3/10; A61K 38/17

(52) U.S. Cl. CPC A23J 3/10; A61K 38/1709

(57) ABSTRACT

The invention provides methods for improving at least one physicochemical property of a product comprising casein micelles, the method comprising applying carbon dioxide to an aqueous product comprising casein micelles to reduce the pH of the product; increasing the pH of the product; and collecting the product comprising casein micelles, in which at least some of the casein micelles of the product are modified in structure by one or more of steps a) and b) compared to naturally occurring casein micelles thereby improving at least one physicochemical property of the product comprising casein micelles. The method reduces viscosity and improves mouth feel of reconstituted product, reduces rehydration time for dried product, improves cheese manufacturing by reducing rennetting or clotting time in cheese manufacture or reducing the amount of rennet required in cheese manufacture.

### FIELD

The present invention relates to improved products containing casein and methods for their production.

### BACKGROUND

Casein is the name for a family of related phosphoproteins (αS1, αS2, β, κ). These proteins are commonly found in mammalian milk, making up 80% of the proteins in cow's milk and between 20% and 45% of the proteins in human milk.

Casein may be extracted from milk and the extraction processes typically produce either acid or rennet forms. Acid casein includes lactic, hydrochloric and sulphuric acid caseins. Caseinates are salt forms of acid caseins and include sodium, potassium and ammonium caseinate.

Casein exists in milk in complex groups of molecules (sometimes referred to as calciumphospho-caseinate) that are called micelles. The micelles consist of casein molecules, calcium, inorganic phosphate and citrate ions, and have a typical molecular weight of several hundred million. In terms of physical chemistry, the casein micelles may be considered to exist in milk as a very stable colloidal dispersion. The caseins, as proteins, are made up of many hundreds of individual amino acids, each of which may have a positive or a negative charge, depending on the pH of the environment. The isoelectric point of casein is approximately 4.6 and it is the pH value at which acid casein is precipitated. In milk, which has a pH of about 6.6, the casein micelles have a net negative charge and are quite stable. Casein is relatively hydrophobic, making it poorly soluble in water. Purified casein is water insoluble and has slow dissolution properties.

Casein can absorb substantial amounts of water so it can modify the texture of dough or baked products, serve as a matrix former in cheese-type products, produce specialised plastics materials or increase the consistency of solutions such as soups. Casein is a good film-former and finds use in whipping and foaming applications and in emulsions of fats or oils in fluids.

Casein is used in several food applications including in baking, cheese products, coffee whiteners and creamers, confectionery, cultured milk products such as yoghurt, high fat powders, shortenings and spreads ice-cream and frozen desserts, infant foods and formula, instant breakfasts and beverages, meat products, nutritional supplements, beverages and bars, pharmaceuticals, soups and gravies, sports drinks and whipped toppings. Casein is generally not consumed as a food on its own—it is used as an ingredient in food products for the purpose of either modifying the physical properties of those food products or providing nutritional supplementation to them. There are also non-food uses of casein, such as in paints and adhesives.

Casein is a major component of milk protein concentrate (MPC), a dairy product with high protein content (40-90 wt %) produced from skim milk by a series of processes that include ultrafiltration (UF), diafiltration (DF), evaporation and drying. MPC contains undenatured forms of both casein and whey protein. The level of protein, lactose and mineral present varies depending on the protein concentration. Ultrafiltration determines the composition of the MPC while evaporation and drying are used to remove water.

Commercially MPCs are available in a range of protein levels from 42% to 85%. MPCs are frequently described with the % dry matter as milk protein being appended to “MPC”. For example MPC85 is an MPC with 85% of the dry matter as milk protein.

MPC is used for its nutritional and functional properties. It is high in protein content and contributes approximately 360 kcal/100 g. Its high protein, low lactose ratio makes MPC an excellent ingredient for protein fortified beverages and foods.

MPC can make products more heat stable and it can provide solubility and dispersibility. This solubility makes MPCs beneficial in dairy-based mixes. MPC is useful in foaming and whipping as the proteins in MPC act at air/water interface to form a stable film of air bubbles. This stabilises certain whipped or foamed products. Proteins in MPC can also act at the oil/water interface to form and stabilise fat emulsions in meat, dairy and baked food products.

Casein and MPC have a wide range of application such as ingredients in the food and beverage industry, for cheese and yoghurt manufacture, confectionary, nutritional and dietary products including protein supplements, aged care products, infant formulas, protein bars, cultured products, frozen desserts, baked goods, and milk powders/creamers. However their uses are somewhat restricted due to poor dissolution properties resulting in slow rehydration.

An example of a common use of milk powders is as a component of a dry beverage mix, such as a meal replacement, protein supplement or infant formula, or in a vending machine. These beverages may be served hot or cold. For this purpose it is desired that milk powders have specific physical properties to avoid such problems as caking of the powder.

In the dairy industry as a whole, powder dissolution or rehydration is considered as a key determinant of overall reconstitution quality and this is a recognised problem with milk powders containing casein, such as MPC, particularly at low temperatures.

Therefore there is a need for milk powders with improved dissolution properties or rehydration properties in fluid, particularly in cold water.

### SUMMARY

A first aspect provides a method for improving at least one physicochemical property of a product comprising casein micelles, the method comprising
a) applying carbon dioxide to an aqueous product comprising casein micelles to reduce the pH of the product;
b) increasing the pH of the product; and
c) collecting the product comprising casein micelles, in which at least some of the casein micelles of the product are modified in structure by one or more of steps a) and b) compared to naturally occurring casein micelles thereby improving at least one physicochemical property of the product comprising casein micelles.

In one embodiment this product of the method is a liquid and the method further comprises drying the product. In one embodiment the product of the method is dried to a powder.

A second aspect provides a product comprising casein micelles, in which at least some of the casein micelles are modified in structure compared to naturally occurring casein micelles, which product is prepared according to the method of the first aspect.

In one embodiment of the second aspect the product of the method is a dry powder.

The inventors have found that the application of carbon dioxide to milk protein concentrate acidifies the MPC. They found that removal of the CO₂ from the chamber restored the pH of MPC to natural levels. On examination of the MPC produced from the method, the inventors determined that structure of the casein micelles in the MPC was modified and that MPC having casein micelles with this modified structure had improved physicochemical properties to MPC with naturally occurring casein micelles, including for the powdered composition, lower viscosity of reconstituted product and concentrate for atomisation, improved rehydration time, reduced rehydration time in cold fluids, and reduced rennetting or clotting time when used in cheese manufacture. Improving the rehydration time and viscosity of MPC may provide improved formulation strategies and improved mouth feel for rehydrated MPC drinks.

Accordingly in an embodiment of the method of the first aspect or product of the second aspect the physiochemical property that the method improves is one or more of reducing viscosity when in liquid form or when reconstituted from dried product, reducing rehydration time for dried product, improving mouth feel, improving cheese manufacturing by reducing rennetting or clotting time in cheese manufacture or reducing the amount of rennet required in cheese manufacture,

A third aspect provides a method for reducing the viscosity of a product containing casein micelles, the method comprising
a) applying carbon dioxide to an aqueous product comprising casein micelles to reduce the pH of the product;
b) increasing the pH of the product; and
c) collecting the product comprising casein micelles, in which at least some of the casein micelles are modified in structure by one or more of steps a) and b) compared to naturally occurring casein micelles,
   which product has reduced viscosity compared to that of the aqueous product comprising casein micelles.

In one embodiment the product has decreased viscosity after drying and reconstitution.

A fourth aspect provides a method for reducing rehydration time for a product containing casein micelles, the method comprising
a) applying carbon dioxide to an aqueous product comprising casein micelles to reduce the pH of the product;
b) increasing the pH of the product; and
c) drying the product to produce a dry product in which at least some of the casein micelles are modified in structure by one or more of steps a) and b) compared to naturally occurring casein micelles,
   which dry product has reduced rehydration time in cold water or milk compared to a dry product made from the starting material.

A fifth aspect provides a method for improving cheese manufacture, the method comprising
a) applying carbon dioxide to an aqueous product comprising casein micelles to reduce the pH of the product;
b) increasing the pH of the product; and
c) collecting the product in which at least some of the casein micelles are modified in structure by one or more of steps a) and b) compared to naturally occurring casein micelles,
   which product reduces the rennetting/clotting time and/or amount of rennet required in cheese manufacture compared to the starting material.

A sixth aspect provides a method for improving at least one physicochemical property of a product comprising casein micelles, the method comprising
a) applying carbon dioxide to an aqueous product comprising casein micelles to reduce the pH of the product;
b) freeze drying the product thereby increasing its pH; and
c) collecting the product comprising casein micelles, in which at least some of the casein micelles of the product are modified in structure by one or more of steps

a) and b) compared to naturally occurring casein micelles thereby improving at least one physicochemical property of the product comprising casein micelles.

The inventors propose that step a) increases the surface area of the casein micelles thus making the freeze drying more efficient and quicker.

A seventh aspect provides a product comprising casein micelles, in which at least some of the casein micelles are modified in structure compared to naturally occurring casein micelles which product is prepared according to the method of the sixth aspect.

In an embodiment of the method of the sixth aspect or product of the seventh aspect the physiochemical property that the method improves is one or more of reducing viscosity when in liquid form or when reconstituted from dried product, reducing rehydration time for dried product, improving cheese manufacturing by reducing rennetting or clotting time in cheese manufacture or reducing the amount of rennet required in cheese manufacture,

An eighth aspect provides a method for increasing the concentration of a product comprising casein micelles to be spray dried comprising
a) applying carbon dioxide to an aqueous product comprising casein micelles to reduce the pH of the product;
b) increasing the pH of the product; and
c) collecting the product in which at least some of the casein micelles are modified in structure by one or more of steps a) and b) compared to naturally occurring casein micelles,
   in which the modified structure of the casein micelles allows for higher concentration of feed solids to be spray dried due to a reduced viscosity.

### FIGURES

In the appended examples, reference will be made to the accompanying figure as follows:

FIG. 1 is a schematic representation of the method of the first aspect.

FIG. 2 plots viscosity (cP) over time (seconds) for reconstituted MPC treated according to the first aspect or conventionally.

FIG. 3 plots particle size (μm) over time (hours and minutes) for MPC treated according to the first aspect or conventionally to show the impact of the method of invention on particle rehydration time.

### DETAILED DESCRIPTION

The inventors sought to provide a product comprising casein which has improved functional properties compared to similar products provided in the prior art.

The inventors determined that by acidification of an aqueous milk product comprising casein micelles (specifically MPC85) using carbon dioxide gas and subsequent removal of that gas and the resultant increase in pH the structure of the casein micelles was modified and the functional properties desired were obtained. While not wishing to be bound by theory the inventors propose that the change in structure of the casein micelle is responsible for the improved physicochemical properties.

The casein micelle is composed of numerous sub-micelles which are held together by colloidal calcium phosphate. It is known that under acidic conditions the colloidal calcium phosphate becomes soluble and therefore the amount of binding between the sub-micelles decreases causing the casein micelle to disassemble. This process can be monitored by measuring the size of protein aggregates using electron microscopy. The inventors propose that the reaction is reversible but that when the pH is increased the colloidal calcium phosphate resumes binding between the sub-micelles but that that the resultant structure of the casein micelle is modified compared to the starting structure.

In one embodiment the product comprising casein micelles is a dairy product such as milk protein concentrate, milk protein isolate, micellar casein, casein, milk protein hydrolysate, whole milk, skim milk, semi-skim milk, buttermilk, cream, cheese powders, yoghurt powders and cheese flavoured powders. The product comprising casein micelles may contain 3%, 6%, 9%, 12%, 15%, 20%, 30%, 40%, 50%, 60%, 70%, 80%, 85%, 90%, 95% or more milk solids.

In a preferred embodiment the product comprising casein micelles is MPC. The MPC may be MPC70, MPC75, MPC80, MPC85 or MPC with other milk solid compositions. Such MPC may be prepared from milk, particularly from skim milk immediately prior to performing the methods of the invention, may be stored in aqueous form prior to performing the methods of the invention, may be reconstituted from dry or powdered MPC or otherwise.

The product may be reduced in certain agents such as lactose and “ash” (which comprises the mineral components of milk) or may comprise an increased concentration of desirable agents.

In one embodiment of the methods of the invention the casein micelle is further manipulated before, simultaneously or subsequent to step a) and/or step b). The further manipulation may involve addition of additives, and complexing agents during step a) when it is proposed that such additive and complexing agents may be taken up within the casein micelle. Other manipulation may include the application of varying heat and/or pressure treatments. The time of manipulation may vary depending upon desired outcomes and the nature of the milk or milk product comprising casein.

The additives and complexing agents include agents which are desirable to be encapsulated within the casein micelle including minerals, vitamins, biologically active agents including basic proteins such as lactoferrin and angiogenin, prebiotics, probiotics, enzymes, flavouring agents, sweeteners, preservatives such as sodium benzoate, potassium sorbate, solvents, buffers and diluents. Suitable additives include vitamins and/or minerals selected from at least one of vitamins A, B1, B2, B3, B5, B6, B11, B12, biotin, C, D, E, H and K and calcium, magnesium, potassium, zinc and iron.

If the product is milk or a milk product the milk can be bovine milk or, due to the relatively similar composition of milk possessed by domestic species it is anticipated that milk from other animals including sheep, goats, horses and buffalo will also provide a suitable source of starting material.

The carbon dioxide may be a gas or a liquid (carbonic acid or super critical liquid).

In a preferred embodiment of the methods of the invention the carbon dioxide is a gas.

In a preferred embodiment the addition of carbon dioxide reduces the pH of the product to 6.4 or less, including 6.3, 6.2. 6.1, 6.0, 5.9, 5.8, 5.7, 5.6, 5.5, 5.4, 5.3, 5.2, 5.1, 5.0, 4.9, 4.8, 4.7 or 4.6 or less. In one embodiment the addition of carbon dioxide reduces the pH to 5.8 to 5.2.

In one embodiment step a) is performed for 2 hours or less, 90 minutes or less, 60 minutes or less, 45 minutes or less, 30 minutes or less, 15 minutes or less, 12 minutes or less, 10 minutes or less, 9 minutes or less, 8 minutes or less, 7 minutes or less, 6 minutes or less, 5 minutes or less, 4 minutes or less, 3 minutes or less, 2 minutes or less, 1 minutes or less or 30 seconds or less.

In one embodiment step the pH is reduced to 6.4 or less for 2 hours or less, 90 minutes or less, 60 minutes or less, 45 minutes or less, 30 minutes or less, 15 minutes or less, 12 minutes or less, 10 minutes or less, 9 minutes or less, 8 minutes or less, 7 minutes or less, 6 minutes or less, 5 minutes or less, 4 minutes or less, 3 minutes or less, 2 minutes or less, 1 minutes or less or 30 seconds or less.

The application of carbon dioxide gas to the product in a chamber allows simultaneous increase in pressure and reduction in pH. The solubility of the carbon dioxide gas is greatest at low temperature and high pressure. Accordingly in one embodiment the methods of the invention are carried out at less than ambient temperature or under greater than atmospheric pressure or both.

In one embodiment the method of either or both of steps a) or b) is performed at less than ambient temperature such as 20° C. or less, 15° C. or less, 10° C. or less, 8° C. or less, 5° C. or less or 4° C. or less.

In one embodiment step a) is performed under greater than atmospheric pressure. The pressure may be increased to 1 to 20 bar, including 1, 2, 3, 4, 5, 6, 7, 8, 9, 0, 11, 12, 13, 14, 15, 16, 17, 18, 19 or 20 bar or higher, preferably 3-13 bar.

In one embodiment step b) is performed under a pressure that is reduced relative to that in step a). If step a) involves carbon dioxide gas, step b) may include a degassing step. It should be appreciated that any degassing step does not require removal of all gas, only a reduction in pressure applied to the product in step b) relative to the pressure applied in step a). In the method described in the eighth aspect the degassing step is effectively achieved by sublimination through freeze drying the product.

Any reference herein to “decreasing” or “reducing” includes a decrease or reduction of 1%, 2%, 5%, 10%, 15%, 20%, 30%, 40%, 50%, 60%, 70%, 80%, 90%, 100%, 200%, 300% or more. Accordingly reference to decreasing the viscosity or rehydration time of a product means that the product has a viscosity or rehydration time that is 1%, 2%, 5%, 10%, 15%, 20%, 30%, 40%, 50%, 60%, 70%, 80%, 90%, 100, 200%, 300% or more less than that of the corresponding product made by conventional means.

As used herein rehydration is intended to mean the addition of a fluid to a dry product. In a preferred embodiment the fluid is water, milk, fruit juice, coffee, tea or alcohol. In a preferred embodiment rehydration is performed using cold fluid, i.e. a fluid at 20° C. or less, 15° C. or less, 10° C. or less, 8° C. or less, 5° C. or less or 4° C. or less.

In one embodiment the method of the invention reduces rehydration time of MPC

In one embodiment the methods comprise increasing the pH of a product in a chamber using carbon dioxide gas and subsequent degassing causing a reduction in pH. This can be performed using a gas injection unit.

Gas injection units have been used in the prior art for milk powder processing and are readily available from engineering companies. However in the prior art gas injection commences after the high pressure pump with degasification occurring at the point of atomisation. This method produces low bulk density powder which is not generally desired. The present method performs the degassing step prior to and drying thus retaining normal bulk density and powder characteristics.

The step of “degassing the chamber” may be performed by an evaporator, vacuum pump, centrifugal separators, hydrocyclone or otherwise. The degassing step does not require removal of all gas from the chamber and includes removal of at least some gas to increase pH and pressure.

The product of the second or seventh aspect may be used as in the preparation of a food substance, a nutraceutical or a pharmaceutical composition. The product preferably has acceptable sensory properties (such as acceptable smell, taste and palatability) and has improved mouth feel and rehydration properties.

Food substances that can utilize the product of the second or seventh aspects include milk powders, particularly as a component of a dry beverage mixture, for example for use in a vending machine, confectionary, infant formula, bakery products, dry mixes (e.g. pancake or biscuit mixes and beverage mixes), dried soups, fermented milk products, ice-cream and frozen dairy desserts, processed cheese and meat products, sports nutrition powders, which increases athletic performance following consumption, and infant food supplements.

The term “nutraceutical” as used herein refers to an edible product isolated or purified from food, which is demonstrated to have a physiological benefit or to provide protection or attenuation of an acute or chronic disease or injury when orally administered.

The nutraceutical may thus be presented in the form of a dietary preparation or supplement, either alone or admixed with edible foods or drinks.

The nutraceutical composition may be in the form of a soluble powder, a liquid or a ready-to-drink formulation. Alternatively, the nutritional composition may be in solid form; for example in the form of a ready-to-eat bar or breakfast cereal. Various flavours, fibres, sweeteners, and other additives may also be present.

The pharmaceutical also comprises one or more pharmaceutically acceptable carriers, diluents or excipients. Such compositions may comprise buffers such as neutral buffered saline, phosphate buffered saline and the like; carbohydrates such as glucose, mannose, sucrose or dextrans; mannitol; proteins; polypeptides or amino acids such as glycine; antioxidants; chelating agents such as EDTA; adjuvants and preservatives.

Products of the second and seventh aspects are preferably formulated for oral consumption. Such a product may be administered to a subject in a manner appropriate to the disease to be treated and/or prevented. The quantity and frequency of administration will be determined by such factors as the condition of the subject and the type and/or severity of the subject's disease. Appropriate dosages may also be determined by clinical trials. An effective amount of the product can be determined by a physician with consideration of individual differences in age, weight, disease severity, condition of the subject, route of administration and any other factors relevant to treatment of the subject. Essentially, an “effective amount” of the product is an amount which is sufficient to achieve a desired therapeutic effect.

Products of the second and seventh aspects may be used in methods for the treatment and/or prevention of diseases. Such treatment methods comprise administering to a subject an effective amount of a nutraceutical or pharmaceutical composition as described above.

Generally products comprising spray dried MPC which is to be rehydrated on use have poor mouth feel (clay-like) due to the large particle size of the MPC and extremely slow rehydration time. Prior art methods to avoid the poor mouth feel include sieving the MPC and using only the smallest particles. This is time consuming and difficult on a commercial scale. The methods of the invention substantially reduce the particle size of the spray dried MPC, allowing for easier formulation and improved mouth feel.

In the present specification, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

As used in the present specification, except where the context requires otherwise due to express language or necessary implication, the term “consisting essentially of” is intended to convey that additional elements may be added but that these additional elements do not materially affect the basic and novel characteristic of the stated feature or features.

As used in the present specification, the singular forms “a”, “an” and “the” include plural aspects unless the context clearly dictates otherwise. Thus, for example, reference to a casein micelle includes one or more casein micelles.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

The invention is now further described in detail by reference to the following example. The example is provided for purposes of illustration only, and is not intended to be limiting unless otherwise specified. Thus, the invention encompasses any and all variations which become evident as a result of the teaching provided herein.

### EXAMPLES

### Example 1

### Standard Preparation of Milk Protein Concentrate

MPCs are produced from skim milk by a pressure driven process known as ultrafiltration (UF) using semipermeable membranes. Lactose and minerals are removed by UF until the desired protein content is reached. The membrane retains the concentrated protein fraction in a fraction referred to as the retentate. The lactose and mineral stream pass through the membrane in a fraction referred to as the permeate.

The retentate is generally spray dried to produce an MPC powder. MPC prepared in this way is commercially available with differing protein content, including MPC85, from Murray Goulburn Cooperative Co. Ltd. (Melbourne, Australia) and various other suppliers. MPC can be either fresh powder or reconstituted from spray dried.

### Example 2

### New Method for Preparation of Milk Protein Concentrate

In certain embodiments, the methods of the invention comprise acidification of milk or an aqueous milk product comprising casein under pressure by applying a gas to the milk in a chamber in an amount capable of reducing the pH of the milk or aqueous milk product to less than 6.4. The chamber is then degassed which increases the pH of the milk or aqueous milk product towards its pre-acidified level and relieves the pressure. The resultant composition comprises casein micelles, in which at least some of the casein micelles are modified in structure compared to naturally occurring casein micelles.

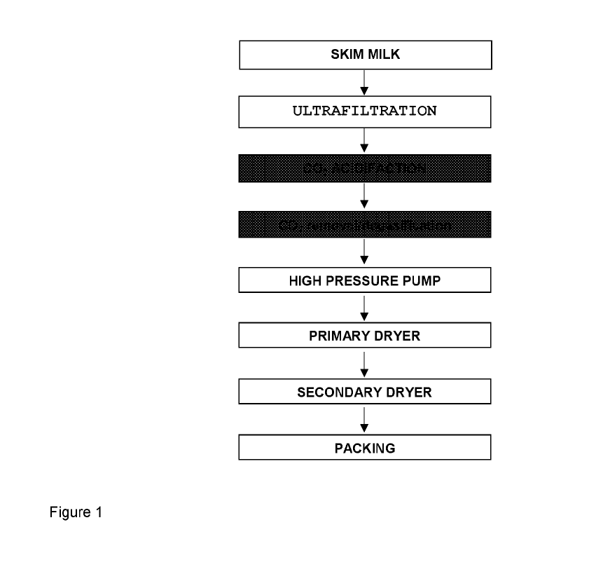

The method is represented schematically in FIG. 1 which includes the step of ultrafiltration which is not essential in the present invention. In the preparation of MPC with altered physicochemical properties in accordance with embodiments of the invention carbon dioxide gas is applied to MPC in a chamber until the pH reduces to 6.4 or less (preferably between 5.2 and 5.8) and the pressure in the chamber is increased (preferably until it reaches 3-13 bar).

After 15 minutes or less under these conditions the chamber is degassed and the resultant composition subjected to high pressure pumping followed by drying and packaging.

The method was performed in duplicate on spray dried MPC85 obtained from Murray Goulburn Cooperative Co. Limited (Melbourne, Australia) reconstituted in water to a concentration of 15% total solids and cooled to 10° C. The resulting products were termed run 1 CO₂ and run 2 CO₂.

The method was also performed on a UF retentate of MPC85 from Murray Goulburn Cooperative Co. Limited (Melbourne, Australia) and the resulting product termed Exp CO₂

### Example 3

### Comparison of Viscosity of Commercially Available MPC and MPC Prepared by Example 2

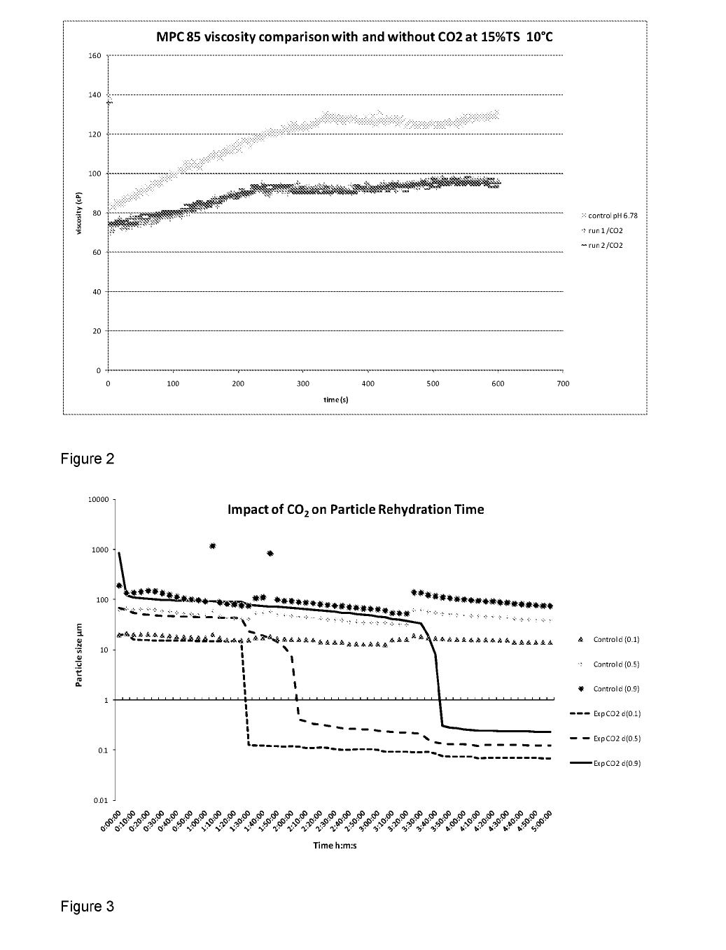

Commercially available spray dried MPC85 powder prepared in accordance with Example 1, obtained from Murray Goulburn Cooperative Co. Limited (Melbourne, Australia), (control) and prepared by the method of Example 2 (run 1 CO₂ and run 2 CO₂) was reconstituted in water to a concentration of 15% total solids and cooled to 10° C. The viscosity was measured over a time period of 10 minutes at 10° C. and a constant stirrer speed of 500 rpm using a Newport Scientific RVA 4 Texture Analyser. The results, represented in FIG. 2, show that the MPC85 made in accordance with the invention has substantially reduced viscosity (70-95 cP) compared to control (80-135 cP) over time.

### Example 4

### Comparison of Rehydration Time of Commercially Available MPC and MPC Prepared by Example 2

The diameter of naturally occurring casein micelles typically fall in the range of 100 nm-300 nm. This diameter can be used as a measure of the level of rehydration. A fully rehydrated MPC powder should have a particle distribution within the range of a naturally occurring casein micelle. Rehydration of spray dried MPC is difficult and is a known problem. The rate at which rehydration occurs can be influenced by temperature and sheer. Typically increasing solvent temperature (>50° C.) and sheer rate are used to rehydrate MPC powders. Rehydration at low temperatures increases the time for particles to revert to original size and generally this can take days.

A Malvern Mastersizer 2000 (Malvern Instruments Ltd UK) with a Hydro 2000G wet dispersion unit was used to measure particle size distribution. MPC85 powder (control) and prepared in accordance with Example 2 (Exp CO₂) from ultrafiltration retentate supplied by Murray Goulburn Cooperative Co. Limited (Melbourne, Australia) had the final powder composition shown in Table 1.

A sample of each powder was added to 20° C. water using a stirrer at 500 rpm at a concentration of 8%. A sample of each solution was then added to dispersion unit until the obscuration was in range 5%-95%. The particle size distribution was measured every 5 minutes. The results, represented in FIG. 3, show that at 3:50:00 (3 hours 50 minutes) mark 90% (d(0.9)) of MPC85 from Example 2 had a particle size within the range of naturally occurring casein micelles (100 nm-300 nm). This is in contrast to the MPC85 (control), assessed using the same methodology, where the powder particle did not significantly reduce in size with rehydration at the given parameters, showing little or no rehydration over 5 hours. Had the experiment been extended very little rehydration would have been expected over 24 hours or more.

TABLE 1

| | Control | Exp CO₂ |
|---|---|---|
| moisture % | 5.1 | 5.5 |
| protein (as is) % | 82.1 | 82.0 |
| fat % | 1.8 | 1.7 |
| ash % | 7.3 | 7.3 |

1.-17. (canceled)

18. A method for reducing the viscosity of a product containing casein micelles, the method comprising
a) applying carbon dioxide to an aqueous product comprising casein micelles to reduce the pH of the product;
b) increasing the pH of the product; and
c) collecting the product comprising casein micelles, in which at least some of the casein micelles are modified in structure by one or more of steps a) and b) compared to naturally occurring casein micelles,
which product has reduced viscosity compared to that of the aqueous product.

19. A method for reducing rehydration time for a product containing casein micelles, the method comprising
a) applying carbon dioxide to an aqueous product comprising casein micelles to reduce the pH of the product;
b) increasing the pH of the product; and
c) drying the product to produce a dry product in which at least some of the casein micelles are modified in structure by one or more of steps a) and b) compared to naturally occurring casein micelles,
which dry product has reduced rehydration time in cold water or milk compared to a dry product made from the starting material.

20. A method for increasing the concentration of a product comprising casein micelles to be spray dried comprising
a) applying carbon dioxide to an aqueous product comprising casein micelles to reduce the pH of the product;
b) increasing the pH of the product; and
c) collecting the product in which at least some of the casein micelles are modified in structure by one or more of steps a) and b) compared to naturally occurring casein micelles,
in which the modified structure of the casein micelles allows for higher concentration of feed solids to be spray dried due to a reduced viscosity

21. The method of claim 18, in which the product comprising casein micelles is a dairy product selected from milk protein concentrate, milk protein isolate, micellar casein, casein, milk protein hydrolysate, whole milk, skim milk, semi-skim milk, buttermilk, cream, cheese powders, yoghurt powders and cheese flavoured powders.

22. The method of claim 18, in which the carbon dioxide is a gas or a liquid.

23. The method of claim 19, in which the carbon dioxide is a gas or a liquid.

24. The method of claim 20, in which the carbon dioxide is a gas or a liquid.

25. The method of claim 18, in which either or both of steps a) or b) is performed at less than ambient temperature.

26. The method of claim 19, in which either or both of steps a) or b) is performed at less than ambient temperature.

27. The method of claim 20, in which either or both of steps a) or b) is performed at less than ambient temperature.

28. The method of claim 18, in which step a) is performed under greater than atmospheric pressure.

29. The method of claim 19, in which step a) is performed under greater than atmospheric pressure.

30. The method of claim 20, in which step a) is performed under greater than atmospheric pressure.

31. The method of claim 18, in which step b) is performed under a pressure that is reduced relative to that in step a).

32. The method of claim 19, in which step b) is performed under a pressure that is reduced relative to that in step a).

33. The method of claim 20, in which step b) is performed under a pressure that is reduced relative to that in step a).

34. A product comprising casein micelles in which at least some of the casein micelles are modified in structure compared to naturally occurring casein micelles, which modified casein micelles are a result of the application of carbon dioxide to an aqueous product comprising casein micelles to reduce the pH of the product and then increasing the pH of the product.

35. The product of claim 34, in which the product comprising casein micelles is a dairy product selected from milk protein concentrate, milk protein isolate, micellar casein, casein, milk protein hydrolysate, whole milk, skim milk, semi-skim milk, buttermilk, cream, cheese powders, yoghurt powders and cheese flavoured powders.

36. A food substance, a nutraceutical composition or a pharmaceutical composition comprising the product of claim 34.
